(19) United States
(12) Patent Application Publication
(10) Pub. No.: US 2018/0003953 A1
(43) Pub. Date: Jan. 4, 2018

(54) CALIBRATION APPARATUS AND METHOD FOR CALIBRATING OPTICAL SCANNING APPARATUS

(71) Applicant: OLYMPUS CORPORATION, Tokyo (JP)

(72) Inventors: Masato FUJIWARA, Tokyo (JP); Yuhei TAKATA, Tokyo (JP)

(73) Assignee: OLYMPUS CORPORATION, Tokyo (JP)

(21) Appl. No.: 15/705,364

(22) Filed: Sep. 15, 2017

Related U.S. Application Data
(63) Continuation of application No. PCT/JP2015/001516, filed on Mar. 18, 2015.

(51) Int. Cl. G02B 26/10; A61B 1/06; A61B 1/00; A61B 1/04; G02B 23/24; G02B 1/11

(52) U.S. Cl. CPC G02B 26/103; G02B 23/2469; G02B 1/11; A61B 1/00057; A61B 1/04; A61B 1/0684

(57) ABSTRACT

This method is for calibrating an optical scanning apparatus that includes an optical fiber with a tip supported to allow vibration and an actuator that drives the tip of the optical fiber in a direction perpendicular to the optical axis of the optical fiber. The method includes arranging a position sensitive detector that detects a position of emitted light from the tip of the optical fiber (step S02) and detecting the position of the emitted light with the position sensitive detector while supplying light to the optical fiber and driving the tip of the optical fiber (step S03). The step of detecting (step S03) is performed using an interference fringe reducer that reduces interference fringes occurring along an optical path reaching the position sensitive detector.

### CROSS-REFERENCE TO RELATED APPLICATION

The present application is a Continuing Application based on International Application PCT/JP2015/001516 filed on Mar. 18, 2015, the content of which is incorporated herein by reference.

### TECHNICAL FIELD

This disclosure relates to a method for calibrating an optical scanning apparatus and to a calibration apparatus.

### BACKGROUND

A known optical scanning apparatus periodically vibrates an optical fiber to scan emitted light over an object. For example, an optical scanning endoscope irradiates an object with illumination light by vibrating an optical fiber with a periodically expanding and contracting amplitude so as to trace a spiral. Light to be detected, such as reflected light and fluorescent light, that is obtained by irradiation of the illumination light is detected at predetermined detection timings. Pixel positions are allocated to the detected signal to generate an image. Therefore, to generate an image with an optical scanning endoscope apparatus, information on the irradiation position of the illumination light at each point in time of the optical scan is necessary. The method for determining the irradiation position of the illumination light uses the elapsed time from the start of vibration of the optical fiber. Since there is individual variation in optical scanning endoscopes, this information on the irradiation position needs to be acquired for each apparatus.

In order to associate the irradiation position of illumination light with the time elapsed from the start of vibration, an optical scanning endoscope is calibrated by using a Position Sensitive Detector (PSD) in advance to acquire the position where the tip of the optical fiber is located at each time. The PSD is a sensor for detecting the position of a light spot formed on a receiving surface. Time-series data on the barycenter of the light spot can be obtained using a PSD. The position at which illumination light is irradiated can thus be associated with the time elapsed after the start of vibration for each optical scanning apparatus.

With position detection by PSD, however, it is known that detection error occurs because of the effect of noise. Therefore, even when calibrating using a PSD, the calculated spot formation position ends up differing from the actual spot formation position because of such detection error. In other words, the calculated spot formation positions do not trace a smooth spiral trajectory, but rather vary. For example, when acquiring an image of an object in which a cross is drawn, the formed image is distorted, as illustrated in FIG. 8.

A method has therefore been proposed to correct the spot formation positions (pixel positions) using polynomial approximation and reduce variation in the calculated spot formation positions due to detection error, thereby reducing image distortion (for example, see JP 2012-147831 A (PTL 1)).

### CITATION LIST

### Patent Literature

PTL 1: JP 2012-147831 A

### SUMMARY

A method according to this disclosure is for calibrating an optical scanning apparatus comprising an optical fiber with a tip supported to allow vibration and an actuator configured to drive the tip of the optical fiber in a direction perpendicular to an optical axis of the optical fiber, the method comprising:

arranging a position sensitive detector configured to detect a position of emitted light from the tip of the optical fiber; and

detecting a position of the emitted light with the position sensitive detector while supplying light to the optical fiber and driving the tip of the optical fiber;

wherein the step of detecting is performed using an interference fringe reducer configured to reduce interference fringes occurring along an optical path reaching the position sensitive detector.

The interference fringe reducer may reduce interference fringes by reducing reflection occurring in the position sensitive detector.

A light-transmitting member for protecting a receiving surface of the position sensitive detector may be disposed opposite the receiving surface, and the interference fringe reducer may place at least one surface of the light-transmitting member in a low reflection state.

Alternatively, a light-transmitting member for protecting a receiving surface of the position sensitive detector may be disposed opposite the receiving surface, and the interference fringe reducer may be configured by a medium filled between the receiving surface of the position sensitive detector and the light-transmitting member, a refractive index of the medium being closer to a refractive index of the light-transmitting member than a refractive index of air is.

The interference fringe reducer may supply low-coherence light to the optical fiber to reduce occurrence of interference fringes in the position sensitive detector.

A calibration apparatus according to this disclosure is for an optical scanning apparatus comprising an optical fiber with a tip supported to allow vibration and an actuator configured to drive the tip of the optical fiber in a direction perpendicular to an optical axis of the optical fiber, the calibration apparatus comprising:

a controller configured to control the actuator;

a position sensitive detector configured to detect a position of emitted light from the tip of the optical fiber;

a memory configured to store calibration data in accordance with position information of the emitted light, the position information being output by the position sensitive detector; and an interference fringe reducer configured to reduce interference fringes occurring along an optical path reaching the position sensitive detector.

The calibration apparatus may further comprise a light-transmitting member opposite a receiving surface of the position sensitive detector to protect the receiving surface, and the interference fringe reducer may be an anti-reflective coating on at least one surface of the light-transmitting member.

Alternatively, the calibration apparatus may further comprise a light-transmitting member opposite a receiving surface of the position sensitive detector to protect the receiving surface, and the interference fringe reducer may be configured by a medium filled between the receiving surface of the position sensitive detector and the light-transmitting member, a refractive index of the medium being closer to a refractive index of the light-transmitting member than a refractive index of air is.

The calibration apparatus may further comprise a light source configured to supply low-coherence light to the optical fiber, and the light source may function as the interference fringe reducer. The low-coherence light source may be an SLD or an LED.

In this disclosure, “calibration” refers to using an instrument such as a position sensitive detector in advance to acquire, at each time, the position of the fiber tip of an optical scanning apparatus that scans a fiber by vibration or the position of an illumination spot of light emitted from the fiber tip. An “optical scanning apparatus” refers to an apparatus that vibrates the tip of an optical fiber so as to scan light over an object, the optical fiber being supported to allow vibration. Examples of optical scanning apparatuses include optical scanning endoscopes, optical scanning microscopes, and optical scanning projectors. The “position sensitive detector” (PSD) is a optical position detector for detecting the position of a light spot on a detection surface.

The “interference fringe reducer” is a component that reduces interference fringes occurring in the PSD. Specific examples include an anti-reflective coating or microstructure formed on the surface of a protective glass in the PSD, a high-refractive index medium filled between the receiving surface and the protective glass of the PSD, and a light source that emits low-coherence light. The “light-transmitting member” is a member with wavelength characteristics allowing transmission of illumination light used for calibration. The light-transmitting member may, for example, be glass or a light-transmitting resin. The “low reflection state” refers to a state in which reflection occurring at the interface of the light-transmitting member is reduced. Examples include a state in which an anti-reflective coating is formed or a state in which a microstructure on the order of the wavelength of light is formed.

### BRIEF DESCRIPTION OF THE DRAWINGS

In the accompanying drawings:

FIG. 1 illustrates the occurrence of interference fringes in a PSD;

FIG. 2 is a block diagram illustrating an optical scanning endoscope connected to a calibration apparatus according to Embodiment 1;

FIG. 3 is a cross-section illustrating the tip of the optical scanning endoscope apparatus in FIG. 2;

FIG. 4A illustrates a side view of the actuator of an optical scanning endoscope apparatus along with an optical fiber for illumination;

FIG. 4B is a cross-section along the A-A line in FIG. 4A;

FIG. 5 is a cross-section of the PSD in FIG. 2;

FIG. 6 is a flowchart illustrating a calibration procedure;

FIG. 7 is a cross-section of the PSD in a calibration apparatus according to Embodiment 2; and

FIG. 8 illustrates an example of image distortion with a conventional calibration method.

### DETAILED DESCRIPTION

Before describing concrete embodiments of this disclosure with reference to the drawings, the technical considerations serving as the foundation for this disclosure is described. To discover the cause of position data error occurring during calibration, we examined the position detection accuracy of a PSD with a Laser Scanning Microscope (LSM) that performs an optical scan using a pair of galvano mirrors. Specifically, we disposed the receiving surface of the PSD near the observation position of the LSM and acquired a microscopic image by scanning the laser. With a scanning mechanism employing the galvano mirrors used in an LSM, the scanning trajectory is accurate and stable. Hence, an image with accurate position information was obtained. As a result, we observed that interference fringes occur inside the PSD and that the light amount of the microscopic image varies by position. Furthermore, we observed two types of interference fringes, one with narrow intervals and one with wide intervals.

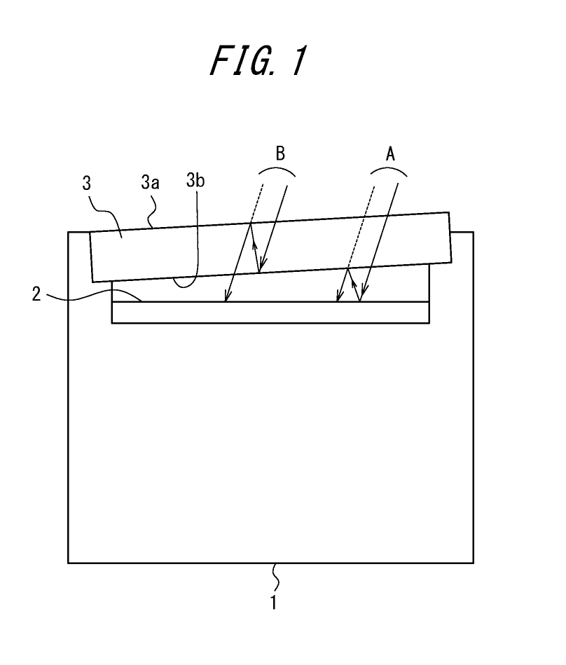

FIG. 1 is a cross-section of the receiving surface of a PSD viewed from the side to illustrate the occurrence of interference fringes in the PSD. A protective glass 3 for protecting a receiving surface 2 of a PSD 1 is disposed opposite the receiving surface 2. The protective glass 3 is slightly inclined with respect to the receiving surface 2 because of manufacturing error. For the sake of explanation, the inclination of the protective glass 3 is emphasized in FIG. 1. The receiving surface 2 of the PSD 1 is a silicon surface that yields strong reflected light with respect to incident light. Interference fringes are thought to occur because of reflection at both sides 3a and 3b of the protective glass 3 in addition to this reflected light. The inclination between the receiving surface 2 and the protective glass is greater than the inclination between the sides 3a, 3b of the protective glass 3. Hence, it is presumed that the interference fringes with narrow intervals are due to the inclination between the receiving surface 2 and the protective glass 3, whereas the interference fringes with wide intervals are due to the sides 3a, 3b of the protective glass 3 being slightly non-parallel.

These results were obtained by observing the receiving surface of the PSD 1 from the LSM side, but interference fringes are thought actually to occur on the receiving surface 2 of the PSD 1 as well. For example, as indicated by A in FIG. 1, interference fringes with narrow intervals occur between light directly incident on the receiving surface 2 and light incident on the receiving surface 2 after being reflected once by the receiving surface 2 and then reflected by the inner surface 3b of the protective glass 3. Also, as indicated by B in FIG. 1, interference fringes with wide intervals occur between light directly incident on the receiving surface 2 and light incident on the receiving surface 2 after being reflected once by each of the outer surface 3a and the inner surface 3b of the protective glass 3. Since the PSD 1 has an appropriate range of the light amount for accurate measurement, output of the light spot position ceases to be accurate if the light amount varies greatly during observation. Therefore, we were convinced that detection error of the PSD 1 is most likely caused by interference fringes due to light reflected at the receiving surface and the protective glass. As described in the embodiments below, we provided means for reducing the interference fringes.

Embodiments of this disclosure are described below with reference to the drawings.

### Embodiment 1

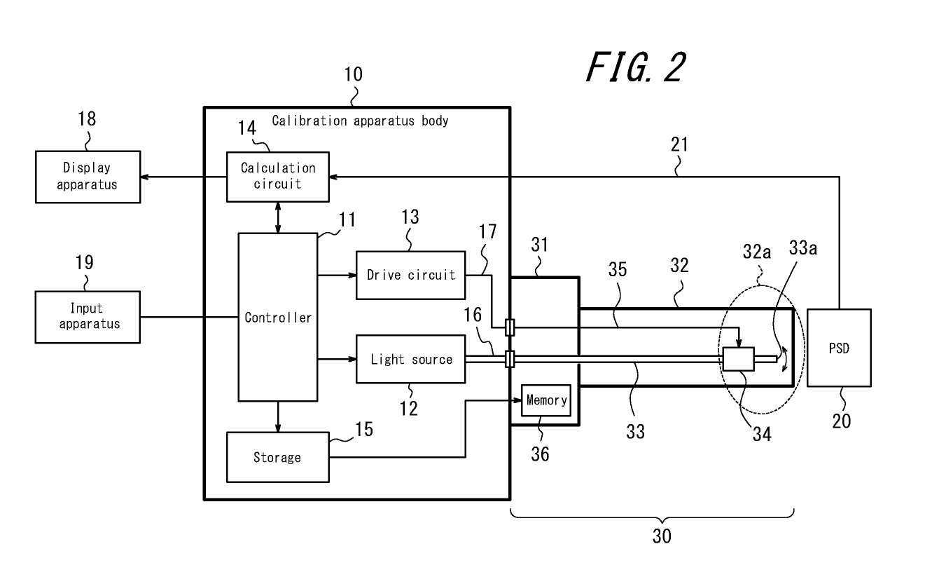

FIG. 2 is a block diagram illustrating an optical scanning endoscope 30 connected to a calibration apparatus according to Embodiment 1. The calibration apparatus includes a calibration apparatus body 10 and a PSD (Position Sensitive Detector) 20. As necessary, a display apparatus 18 such as a display and an input apparatus 19 such as a keyboard, mouse, and/or touch panel are connected to the calibration apparatus body 10. The optical scanning endoscope 30 is connected to the calibration apparatus body 10 by a connector 31.

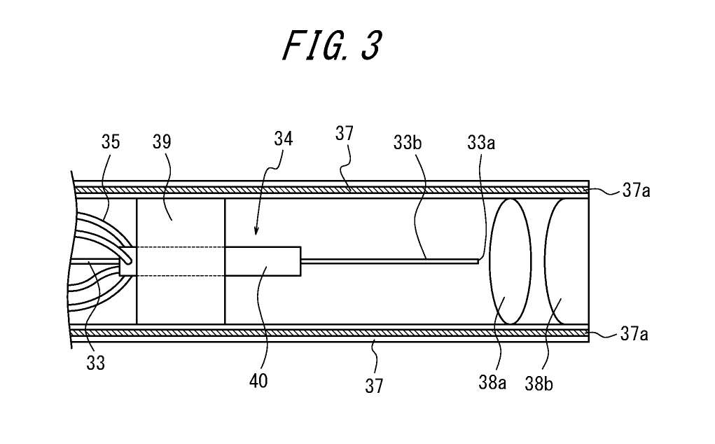

The optical scanning endoscope 30 to be calibrated includes an optical fiber 33 for illumination that is the scope portion of the endoscope apparatus and is inserted through the inside of the optical scanning endoscope 30, an actuator 34 that drives a tip 33a of the optical fiber 33 for illumination, a drive signal wire 35 that transmits a drive signal to the actuator 34, and a memory 36 embedded inside the optical scanning endoscope 30 (for example, in the connector 31). Optical fibers 37 for receiving light (see FIG. 3) are inserted through the optical scanning endoscope 30 and receive and propagate light to be detected, such as reflected light and fluorescent light yielded by irradiation of illumination light.

During endoscopic observation, the optical scanning endoscope 30 is connected to a non-illustrated control apparatus body of an optical scanning endoscope apparatus by the connector 31 and is used to generate an endoscopic image. The optical scanning endoscope apparatus body includes components such as a light source that supplies light to the optical scanning endoscope 30, a drive circuit for driving the actuator 34, and an image processing circuit that generates an image from pixel data received by the optical scanning endoscope 30. Such an optical scanning endoscope apparatus is, for example, disclosed in JP 2014-44265 A and JP 2014-145941 A. The optical scanning endoscope 30 is usually commercially distributed separately from the control apparatus body. The calibration apparatus of this disclosure is mainly for calibrating the optical scanning endoscope 30 at the time of product shipment.

Like the control apparatus body during endoscopic observation, the calibration apparatus body 10 is configured to be connectable to the connector 31 of the optical scanning endoscope 30. The calibration apparatus body 10 includes a controller 11 that controls the calibration apparatus body 10 overall, a light source 12 that supplies illumination light for calibration to the optical scanning endoscope 30, a drive circuit 13 that drives the actuator 34 of the optical scanning endoscope 30, a calculation circuit 14 that receives and processes output from the PSD 20, and a storage 15 that stores calibration data output from the calculation circuit 14.

The light source 12 includes a light source for calibration, such as a laser diode or a Diode-Pumped Solid-State (DPSS) laser. When observing with the optical scanning endoscope 30, a plurality of light sources that emit light of different wavelengths may be used to obtain a color image, but it suffices for the calibration apparatus body 10 to include at least one light source for calibration. The light emission timing of the light source 12 is controlled by the controller 11. Light emitted from the light source 12 is incident on the optical fiber 16 for illumination and combined in the connector 31 onto the optical fiber 33 for illumination of the optical scanning endoscope 30. Single-mode optical fibers may be used as the optical fibers 16, 33 for illumination.

The drive circuit 13 supplies a similar drive signal to the actuator 34 of the optical scanning endoscope 30 as during endoscopic observation. As described below, when the tip of the optical fiber 33 for illumination is driven by piezoelectric elements, the drive circuit 13 supplies driving voltage to the piezoelectric elements. The output of the drive circuit 13 is supplied to the drive signal wire 17. The drive signal wire 17 is connected to the drive signal wire 35 of the optical scanning endoscope 30 by the connector 31. The timing at which driving of the drive circuit 13 starts is also controlled by the controller 11.

Over a detection signal wire 21, the calculation circuit 14 acquires a detection signal that is output by the PSD 20 and corresponds to the illumination position of illumination light on the receiving surface. The calculation circuit 14 then converts the signal to the coordinates (x, y) of the illumination position. Furthermore, the calculation circuit 14 associates the converted coordinates (x, y) with the time elapsed from the start of driving of the drive circuit 13 by the controller 11. As necessary, error in the coordinate positions of illuminated light in the calculation circuit 14 may be smoothed with a technique such as polynomial approximation. The illumination position information, on the illumination light, that is calculated and associated with the elapsed time by the calculation circuit 14 is stored in the storage 15 as calibration data.

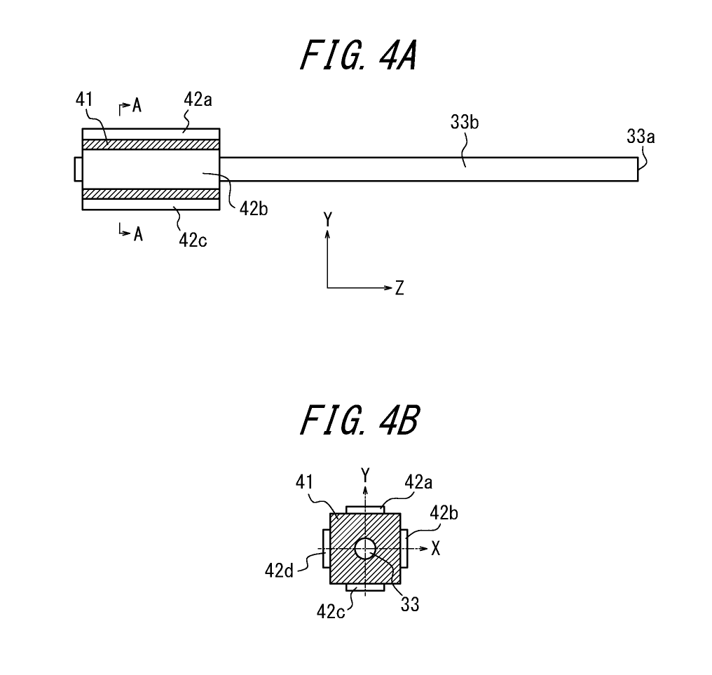

Next, the driving mechanism of the optical scanning endoscope 30 is described. FIG. 3 is a cross-section of the tip 32a of an insertion part 32 of the optical scanning endoscope 30 in FIG. 2 (the portion indicated by the dotted line). The tip 32a of the insertion part 32 of the optical scanning endoscope 30 includes the actuator 34, projection lenses 38a and 38b, the optical fiber 33 for illumination that passes through the central portion, and a plurality of optical fibers 37 for receiving light that pass through the peripheral portion. The optical fibers 37 for receiving light are used to detect the light to be detected during endoscopic observation and are not used for calibration. The actuator 34 includes an actuator tube 40 fixed to the inside of the insertion part 32 by an attachment ring 39, a fiber holding member 41 disposed inside the actuator tube 40, and piezoelectric elements 42a to 42d (see FIGS. 4A and 4B).

The optical fiber 33 for illumination is supported by the fiber holding member 41, and the portion from the fiber holding member 41 to the tip 33a is an oscillating part 33b that is supported to allow vibration. The projection lenses 38a, 38b are disposed at the extreme end of the insertion part 32. The projection lenses 38a and 38b are configured so that laser light emitted from the tip 33a of the optical fiber 33 for illumination is roughly concentrated on the object for observation. Accordingly, the PSD 20 is positioned so that the receiving surface matches the position of concentration. The projection lenses are not limited to a double lens structure and may be structured as a single lens or as three or more lenses.

FIGS. 4A and 4B illustrate the actuator 34 of the optical scanning endoscope 30 along with the optical fiber 33 for illumination, where FIG. 4A is a side view, and FIG. 4B is a cross-section along the A-A line in FIG. 4A. The optical fiber 33 for illumination passes through the center of the fiber holding member 41, which has a prismatic shape, and is thereby firmly held by the fiber holding member 41. The four sides of the fiber holding member 41 respectively face the +Y, −Y, +X, and −X directions, which are perpendicular to the +Z direction that is the optical axis direction of the optical fiber. A pair of piezoelectric elements 42a and 42c for driving in the Y direction are fixed onto the fiber holding member 41 in the +Y direction and the −Y direction, and a pair of piezoelectric elements 42b and 42d for driving in the X direction are fixed in the +X direction and the −X direction. One of the piezoelectric elements 42b and 42d disposed opposite each other with the fiber holding member 41 therebetween expands and the other contracts, thereby causing the fiber holding member 41 to flex. Repeating this operation produces vibration in the X direction. The same is true for vibration in the Y direction as well.

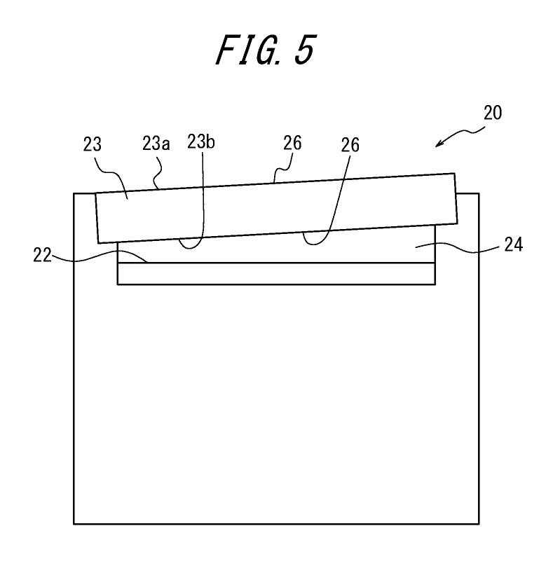

The drive circuit 13 can perform vibration driving of the piezoelectric elements 42b and 42d for driving in the X direction and the piezoelectric elements 42a and 42c for driving in the Y direction by applying vibration voltage of the same frequency or vibration voltage of different frequencies thereto. Upon vibration driving of the piezoelectric elements 42a and 42c for driving in the Y direction and the piezoelectric elements 42b and 42d for driving in the X direction, the oscillating part 33b of the optical fiber 33 for illumination illustrated in FIGS. 3, 4A, and 4B vibrates. The tip 33a thus vibrates and is deflected, so that the laser light emitted from the tip 33a scans the receiving surface 22 of the PSD 20 (see FIG. 5). A spiral scan can be achieved by applying vibration voltages in the X and Y directions with the same frequency, a phase differing by approximately 90°, and an amplitude that varies between zero and the maximum.

Next, the PSD 20 used in this embodiment is described. FIG. 5 is a cross-section of the PSD 20 in FIG. 2 in a direction along the receiving surface 22 (a direction approximately perpendicular to the optical path of light incident on the center of the receiving surface 22). In FIG. 5, the illumination light emitted from the optical fiber 33 for illumination is incident from above. In other words, the PSD 20 of FIG. 2 is disposed with the receiving surface 22 of the PSD 20 in FIG. 5 facing to the left. The PSD 20 includes the receiving surface 22 and a protective glass 23 disposed apart from and facing the receiving surface 22. The receiving surface 22 is formed on a silicon substrate and normally has a high reflectance. An air layer 24 is formed between the receiving surface 22 and the protective glass 23. It is difficult to arrange the receiving surface 22 and the protective glass 23 accurately in parallel, and the protective glass 23 has an undesired inclination relative to the receiving surface 22 (the inclination being emphasized in FIG. 5). The protective glass 23 itself also has a slight wedge angle, so that the outer surface 23a that faces the tip 32a of the optical fiber 33 for illumination and the inner surface 23b that faces the receiving surface 22 of the PSD 20 are not completely parallel.

An Anti-Reflective (AR) coating 26 (an interference fringe reducer) is formed on at least one of the outer surface 23a and the inner surface 23b of the protective glass 23. The AR coating 26 is preferably formed on both the outer surface 23a and the inner surface 23b. The AR coating 26 reduces interference between light reflected by at least one of the surfaces 23a and 23b of the protective glass 23 and illumination light directly incident on the receiving surface 22. In other words, the AR coating 26 functions as an interference fringe reducer that reduces interference fringes occurring along the optical path reaching the PSD 20.

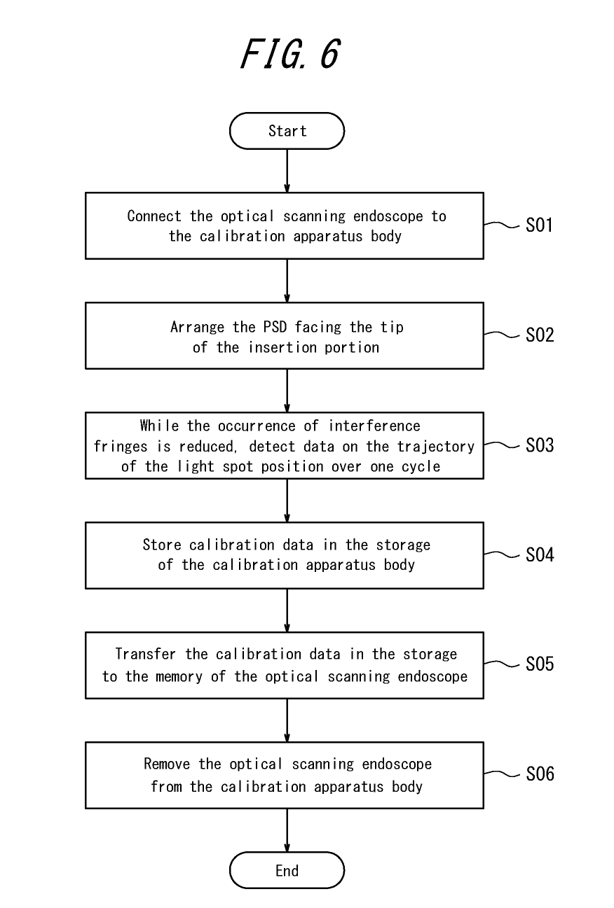

Next, using the flowchart in FIG. 6, the calibration procedure is described. First, in order to perform calibration, the user of the calibration apparatus connects the connector 31 of the optical scanning endoscope 30 to the calibration apparatus body 10 as illustrated in FIG. 2 (step S01). As a result, the optical fiber 16 for illumination and the drive signal wire 17 of the calibration apparatus body 10 are respectively connected to the optical fiber 33 for illumination and the drive signal wire 35 of the optical scanning endoscope 30.

Next, the user fixes the tip 32a of the insertion part 32 of the optical scanning endoscope 30 and arranges the PSD 20 so that the receiving surface 22 of the PSD 20 matches the surface of concentrated light where the illumination light irradiated from the tip 32a forms a spot (step S02). The detection signal wire 21 of the PSD 20 is connected to the calculation circuit 14 of the calibration apparatus body 10.

The user then turns the calibration apparatus body 10 on. Via the input apparatus 19, the user instructs the controller 11 to start calibration. While emitting illumination light with the light source 12, the controller 11 turns on the actuator 34 with the drive circuit 13 and starts a spiral scan. Over at least one cycle of the spiral scan, the PSD 20 sequentially detects the trajectory traced by the spot position of the illumination light (step S03).

Since an AR coating 26 is formed on at least one of the outer surface 23a and the inner surface 23b of the protective glass 23 in the PSD 20, the occurrence of interference fringes on the receiving surface 22 is reduced. As a result, the amount of illumination light incident on the PSD does not vary by scanning position, allowing the spot position of the illumination light to be detected accurately.

The PSD 20 outputs a detection signal corresponding to the detected light spot position (for example, the voltage corresponding to the spot position) sequentially to the calculation circuit 14. The calculation circuit 14 receives information on the time elapsed after activation of the drive circuit 13 from the controller 11 and creates calibration data by associating coordinate information on the spot position calculated from the detection signal with time information. Furthermore, in some cases, the calculation circuit 14 executes processing such as correcting the detection error of the PSD 20, smoothing the detected trajectory, and detecting abnormal values in the data. The calibration data calculated by the calculation circuit 14 is stored in the storage 15 within the calibration apparatus body 10 (step S04). The storage 15 can be a storage apparatus inside the calibration apparatus body 10. Alternatively, the storage 15 may be a portable storage medium, such as a memory card, that can be detached from the calibration apparatus body 10.

Next, once storage of the calibration data in the storage 15 is complete, the calibration data in the storage 15 is output to the memory 36 inside the optical scanning endoscope 30 (step S05). When the storage 15 is a portable storage medium, the user removes the storage 15 from the calibration apparatus body 10 and inserts the storage 15 at a predetermined location on the optical scanning endoscope 30 as the memory 36. In this case, the operation to remove the portable storage medium may be performed after completion of the following step S06.

After the calibration data is output to the memory 36 inside the optical scanning endoscope 30, the user removes the connector 31 of the optical scanning endoscope 30 from the calibration apparatus body 10 (step S06).

In this way, the optical scanning endoscope 30 holds calibration data in the memory 36. During endoscopic observation using the optical scanning endoscope 30, the optical scanning endoscope 30 is connected to a control apparatus body that includes a light source, a drive circuit, and image processor. The control apparatus body reads calibration data from the memory 36 of the optical scanning endoscope 30 and uses the calibration data to associate the acquired pixel values with pixel positions and generate an image.

According to this disclosure, by performing calibration with the aforementioned calibration procedure using the aforementioned calibration apparatus, highly accurate calibration data is stored in the optical scanning endoscope 30. In particular, since an AR coating 26 is formed on at least one of the outer surface 23a and the inner surface 23b of the protective glass 23, interference fringes on the receiving surface 22 can be reduced, thereby maintaining the accuracy of the output of the PSD 20. As a result, during endoscopic observation using the optical scanning endoscope 30, an image with less distortion can be generated. An increase in the accuracy of diagnosis using the optical scanning endoscope 30 can therefore be expected.

Conventionally, it has been difficult to determine whether distortion occurring in an endoscopic image is due to noise within the PSD or to foreign matter, such as dust, on the lens or PSD. After performing the calibration of this disclosure, however, interference fringes within the PSD are reduced, making it easier to distinguish foreign matter on the lens or the PSD. Another effect of performing the calibration method of this disclosure is that image distortion occurring over a large area can be removed, which is difficult to achieve with correction by approximation. Therefore, in the case of local distortion due to the aforementioned dust or the like, the accuracy of correction by approximation improves.

In the optical scanning endoscope 30, the tip 32a of the insertion part 32 that performs optical scanning is extremely small, making it difficult to arrange the sensor or other device that detects the position of the tip 33a of the optical fiber 33 for illumination at the tip. Therefore, the calibration method of this device is particularly suitable when applied to the optical scanning endoscope 30.

### Embodiment 2

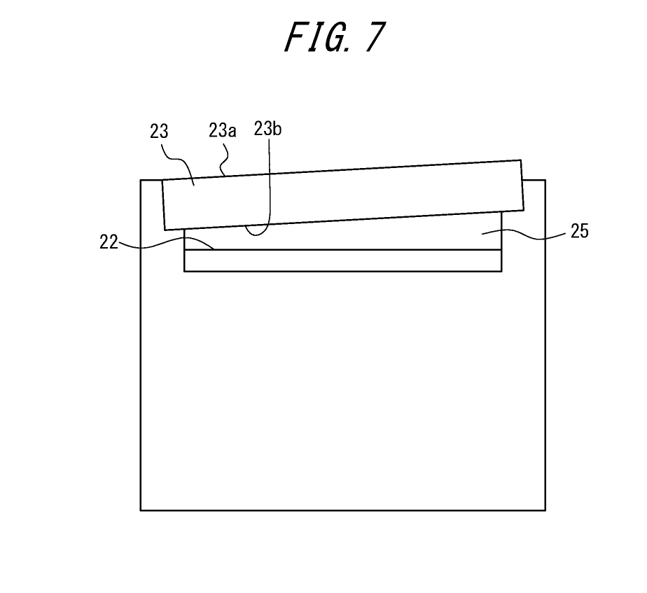

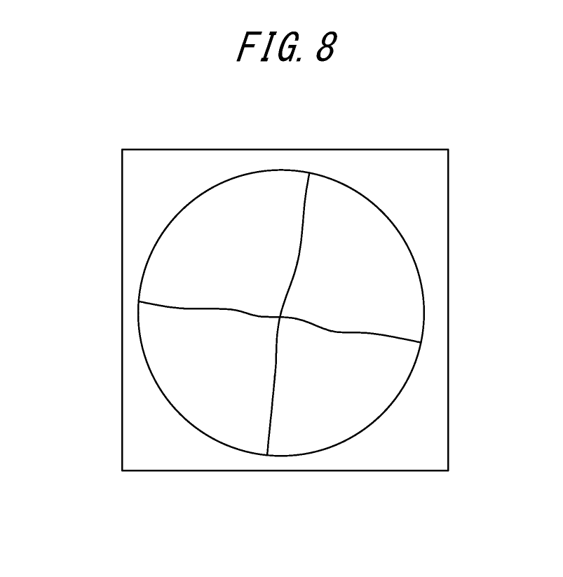

FIG. 7 is a cross-section of the PSD 20 in a calibration apparatus according to Embodiment 2. In Embodiment 2, instead of forming an AR coating on the protective glass 23, the space between the receiving surface 22 and the protective glass 23 of the PSD 20 is filled with a medium 25 (an interference fringe reducer), such as gel, having a refractive index closer to the refractive index of the protective glass than the refractive index of air is. As a result, the difference between the refractive indices of the protective glass 23 and the medium 25 is reduced below the difference between the refractive indices of the protective glass 23 and air, thereby reducing the occurrence of reflected light. The refractive index of the medium 25 is preferably closer to the refractive index of the protective glass 23. In particular, by matching the refractive index of the medium 25 to the refractive index of the protective glass 23, reflection at the interface between the protective glass 23 and the medium 25 can be prevented. Since the remaining configuration is similar to Embodiment 1, identical or corresponding constituent elements are labeled with the same reference signs, and a description thereof is omitted.

According to this embodiment, by reducing the reflection between the protective glass 23 and the medium 25, the occurrence of interference fringes on the receiving surface 22 is reduced. As a result, as in Embodiment 1, an image with less distortion can be generated during endoscopic observation using the optical scanning endoscope 30.

### Embodiment 3

Instead of the PSD 20 in Embodiment 1, Embodiment 3 uses a PSD similar to the PSD 1 with no AR coating in FIG. 1. Also, Embodiment 3 uses a light source 12 with low coherence (an interference fringe reducer), such as a Super Luminescent Diode (SLD) or an LED, for the light source 12 in FIG. 2. The remaining configuration is similar to that of Embodiment 1, and a description thereof is omitted.

According to this embodiment, light with low coherence is used as the illumination light. Therefore, interference fringes are less likely to occur in the PSD 20. As a result, as in Embodiment 1, an image with less distortion can be generated during endoscopic observation using the optical scanning endoscope 30.

While use of a PSD without an AR coating has been described, a PSD with an AR coating 26 like the PSD 20 may be used. In this case, interference fringes can be further reduced by the effects of both the AR coating 26 and the low-coherence light source, increasing the overall effect.

The present disclosure is not limited to the above embodiments, and a variety of changes and modifications may be made. For example, the scanning trajectory of the optical scanning apparatus is not limited to a spiral scan. This disclosure may also be applied to a raster scan or a Lissajous scan. Also, the method for driving the optical fiber of the optical scanning apparatus is not limited to a method using piezoelectric elements. The actuator of the optical fiber tip may instead be configured with an electromagnetic driving method that uses coils and a permanent magnet. In this case, the drive circuit controls the current instead of controlling the voltage applied to the actuator.

Furthermore, in the above embodiment, the controller, light source, drive circuit, calculation circuit, and storage are housed in the same calibration apparatus body, but these components may be separate hardware instead. The material protecting the receiving surface of the PSD has been described as glass, but a different material may be used, such as light-transmitting resin. The above embodiment is applied to calibration before shipping an optical scanning apparatus but may instead be applied to calibration of an optical scanning apparatus already in use. Furthermore, in the above embodiment, a calibration apparatus body exclusively for calibration is provided separately from the control apparatus body for observation, but the functions of the calibration apparatus body may be embedded in the control apparatus body for observation, so that the user can perform calibration at any time.

The optical scanning apparatus is not limited to an optical scanning endoscope and may also be applied to an optical scanning microscope or an optical scanning projector that scans a fiber. In the above embodiments, the optical scanning endoscope that is an optical scanning apparatus does not include a light source and a drive circuit, but this disclosure may also be applied to an optical scanning apparatus that incorporates these components. In this case, the calibration apparatus at least includes the controller of the calibration apparatus body connected to the optical scanning apparatus, the PSD in which the occurrence of interference fringes is reduced, and the storage apparatus storing the position information detected by the PSD. It then suffices to drive the optical scanning apparatus under control of the controller, to detect the position of the optical spot using the PSD, and to store the position in the storage apparatus in association with the timing information. A variety of other modifications may be made to the configuration of the calibration apparatus.

Furthermore, a storage is provided in the calibration apparatus body in the above embodiments, and the calibration data is temporarily stored in the storage. Alternatively, the storage may be omitted from the calibration apparatus body, and the calibration data calculated by the calculation circuit may be output directly to the memory of the optical scanning apparatus.

### INDUSTRIAL APPLICABILITY

The calibration apparatus and the method for calibration according to this disclosure can be used to calibrate an optical scanning apparatus and are particularly suitable for use in calibration before product shipment.

### REFERENCE SIGNS LIST

10 Calibration apparatus body
11 Controller
12 Light source
13 Drive circuit
14 Calculation circuit
15 Storage
16 Optical fiber for illumination
17 Drive signal wire
18 Display apparatus
19 Input apparatus
20 PSD
21 Detection signal wire
22 Receiving surface
23 Protective glass
23a Outer surface
23b Inner surface
24 Air layer
25 Medium
26 Anti-Reflective (AR) coating
30 Optical scanning endoscope
31 Connector
32 Insertion part
32a Tip
33 Optical fiber for illumination
33a Tip
33b Oscillating part
34 Actuator
35 Drive signal wire
36 Memory
37 Optical fiber for receiving light
37a Tip
38a, 38b Projection lens
39 Attachment ring
40 Actuator tube
41 Fiber holding member
42a, 42b, 42c, 42d Piezoelectric element

1. A method for calibrating an optical scanning apparatus comprising an optical fiber with a tip supported to allow vibration and an actuator configured to drive the tip of the optical fiber in a direction perpendicular to an optical axis of the optical fiber, the method comprising:
arranging a position sensitive detector configured to detect a position of emitted light from the tip of the optical fiber; and
detecting a position of the emitted light with the position sensitive detector while supplying light to the optical fiber and driving the tip of the optical fiber;
wherein the step of detecting is performed using an interference fringe reducer configured to reduce interference fringes occurring along an optical path reaching the position sensitive detector.

2. The method of claim 1, wherein the interference fringe reducer reduces interference fringes by reducing reflection occurring in the position sensitive detector.

3. The method of claim 2, wherein a light-transmitting member for protecting a receiving surface of the position sensitive detector is disposed opposite the receiving surface, and the interference fringe reducer places at least one surface of the light-transmitting member in a low reflection state.

4. The method of claim 2, wherein a light-transmitting member for protecting a receiving surface of the position sensitive detector is disposed opposite the receiving surface, and the interference fringe reducer is configured by a medium filled between the receiving surface of the position sensitive detector and the light-transmitting member, a refractive index of the medium being closer to a refractive index of the light-transmitting member than a refractive index of air is.

5. The method of claim 1, wherein the interference fringe reducer supplies low-coherence light to the optical fiber to reduce occurrence of interference fringes in the position sensitive detector.

6. A calibration apparatus for an optical scanning apparatus comprising an optical fiber with a tip supported to allow vibration and an actuator configured to drive the tip of the optical fiber in a direction perpendicular to an optical axis of the optical fiber, the calibration apparatus comprising:
a controller configured to control the actuator;
a position sensitive detector configured to detect a position of emitted light from the tip of the optical fiber;
a memory configured to store calibration data in accordance with position information of the emitted light, the position information being output by the position sensitive detector; and
an interference fringe reducer configured to reduce interference fringes occurring along an optical path reaching the position sensitive detector.

7. The calibration apparatus of claim 6, further comprising a light-transmitting member opposite a receiving surface of the position sensitive detector to protect the receiving surface, wherein the interference fringe reducer is an anti-reflective coating on at least one surface of the light-transmitting member.

8. The calibration apparatus of claim 6, further comprising a light-transmitting member opposite a receiving surface of the position sensitive detector to protect the receiving surface, wherein the interference fringe reducer is configured by a medium filled between the receiving surface of the position sensitive detector and the light-transmitting member, a refractive index of the medium being closer to a refractive index of the light-transmitting member than a refractive index of air is.

9. The calibration apparatus of claim 6, further comprising a light source configured to supply low-coherence light to the optical fiber, wherein the light source functions as the interference fringe reducer.

10. The calibration apparatus of claim 9, wherein the low-coherence light source is an SLD or an LED.
